(19) United States
(12) Patent Application Publication
(10) Pub. No.: US 2024/0201588 A1
(43) Pub. Date: Jun. 20, 2024

(54) ORGANIC SALT, PHOTORESIST COMPOSITION INCLUDING THE SAME, AND PATTERN FORMATION METHOD USING THE SAME

(71) Applicant: Samsung Electronics Co., Ltd., Suwon-si (KR)

(72) Inventors: Hana KIM, Suwon-si (KR); Beomseok KIM, Suwon-si (KR); Kyuhyun IM, Suwon-si (KR); Yoonhyun KWAK, Suwon-si (KR); Hyeran KIM, Suwon-si (KR)

(73) Assignee: Samsung Electronics Co., Ltd., Suwon-si (KR)

(21) Appl. No.: 18/192,316

(22) Filed: Mar. 29, 2023

(30) Foreign Application Priority Data
Nov. 11, 2022 (KR) 10-2022-0150966

(51) Int. Cl. G03F 7/004; C07C 65/10; C07C 309/12; C07C 311/09; C07C 391/02; C07C 395/00; G03F 7/038; G03F 7/039

(52) U.S. Cl. CPC G03F 7/0045; C07C 65/10; C07C 309/12; C07C 311/09; C07C 391/02; C07C 395/00; G03F 7/0382; G03F 7/0397; C07C 2603/74

(57) ABSTRACT

Provided are an organic salt represented by Formula 1, a photoresist composition including the same, and a pattern method using the same:

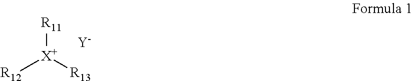

wherein X⁺, Y⁻, and R₁₁ to R₁₃ in Formula 1 are understood by referring to the specification.

### CROSS-REFERENCE TO RELATED APPLICATION

This application is based on and claims priority under 35 U.S.C. § 119 to Korean Patent Application No. 10-2022-0150966, filed on Nov. 11, 2022, in the Korean Intellectual Property Office, the disclosure of which is incorporated by reference herein in its entirety.

### BACKGROUND

### 1. Field

The disclosure relates to an organic salt, a photoresist composition including the same, and a pattern formation method using the same.

### 2. Description of the Related Art

In semiconductor manufacturing, photoresists, whose physical properties change in response to light, are used to form fine patterns. From among these photoresists, chemically amplified photoresists have been widely used. In the case of chemically amplified photoresists, an acid formed by a reaction between light and a photoacid generator reacts again with a base resin to change the solubility of the base resin with respect to a developing solution, thereby enabling finer patterning.

For example, in the case of using a high energy ray having relatively very high energy, such as extreme ultraviolet (EUV), the number of photons is relatively small even when light having the same energy is irradiated. Accordingly, there is a demand for a photoacid generator that can act effectively even when used in a small amount and can provide improved sensitivity and/or resolution.

In addition, in the case of a chemically amplified photoresist, the formed acid diffuses to an unexposed area, and thus, the pattern uniformity is reduced or the surface roughness is increased. Accordingly, there is a need for quenchers with improved dispersibility and/or improved compatibility with base resins.

### SUMMARY

Provided are an organic salt, a photoresist composition including the same, and a pattern formation method using the same, wherein the organic salt may act as a photoacid generator capable of providing improved sensitivity and/or resolution and a quencher having improved dispersibility and/or compatibility.

Additional aspects will be set forth in part in the description which follows and, in part, will be apparent from the description, or may be learned by practice of the presented embodiments of the disclosure.

According to an aspect of the disclosure, an organic salt represented by Formula 1 is provided:

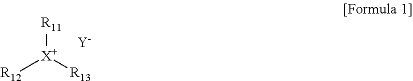

wherein, in Formula 1, X⁺ may be Se⁺ or Te⁺; R₁₁ to R₁₃ may each independently be a linear, branched or cyclic C₁-C₂₀ monovalent hydrocarbon group which may optionally include a heteroatom, and at least one of R₁₁ to R₁₃ may include at least one iodine (I), and two adjacent of R₁₁ to R₁₃ may be optionally bonded to each other to form a ring; and Y⁻ may be a counter anion.

According to another aspect of the disclosure, a photoresist composition includes the organic salt, an organic solvent, and a base resin.

According to another aspect of the disclosure, a pattern formation method includes forming a photoresist film by applying the photoresist composition; exposing at least a portion of the photoresist film to a high energy ray; and developing the exposed photoresist film using a developing solution.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 is a flowchart illustrating a pattern formation method according to some example embodiments; and

FIGS. 2A, 2B and 2C are side cross-sectional views used to describe a pattern formation method according to some example embodiments.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects. As used herein, the term “and/or” includes any and all combinations of one or more of the associated listed items. Expressions such as “at least one of,” when preceding a list of elements, modify the entire list of elements and do not modify the individual elements of the list.

The present disclosure may undergo various modifications and may have various embodiments. Accordingly, specific embodiments are illustrated in the drawings and described in detail in the detailed description. However, this is not intended to limit the present disclosure to a specific embodiment, and should be understood to include all modifications, equivalents, or substitutes included in the spirit and technical scope of the present disclosure. In describing the present disclosure, when it is determined that a detailed description of related known technologies may make the gist of the present disclosure unclear, the detailed description will be omitted.

The terms “first”, “second”, “third”, etc. may be used to describe various elements, but are used only for the purpose of distinguishing one element from another element, and the order or type of the elements are not limited.

Throughout this specification, a portion of a layer, film, region, plate, etc., described as being “on” or “above” another portion thereof may be positioned directly above, below, to the left or right of, while in contact, as well as above, below, to the left or light of, while in a non-contact

Singular expressions include plural expressions unless the context clearly dictates otherwise. Terms such as “include” or “have” are intended to indicate the presence of features, numbers, steps, operations, elements, parts, components, materials, or combinations thereof described in the specification unless otherwise stated, and it should be understood that the terms do not preclude the possibility of the presence or addition of one or more other features, numbers, steps, operations, elements, parts, components, materials, or combinations thereof.

Whenever a range of values is recited, that range includes all values that fall within that range, as if explicitly written out, and further includes the boundaries of the range. Thus, the range of “X to Y” includes all values between X and Y, and al so includes X and Y. When the terms “about” are used in this specification in connection with a numerical value, it is intended that the associated numerical value includes a manufacturing tolerance (e.g., ±10%) around the stated numerical value. Further, regardless of whether values are modified as “about” or “substantially,” it will be understood that these values should be construed as including a manufacturing or operational tolerance (e.g., ±10%) around the stated values.

The term “Cₓ-C_{y}” used herein refers to a case where the number of carbons constituting the substituent is x to y. For example, “C₁-C₆” refers to a case where the number of carbons constituting the substituent is 1 to 6, and “C₆-C₂₀” refers to a case where the number of carbons constituting the substituent is 6 to 20.

The term “monovalent hydrocarbon group” used herein may include, for example, a linear or branched alkyl group (for example, a methyl group, an ethyl group, a propyl group, an isopropyl group, a butyl group, an isobutyl group, a sec-butyl group, a tert-butyl group, a pentyl group, a neopentyl group, a hexyl group, a heptyl group, a 2-ethylhexyl group, and a nonyl group); a monovalent saturated cycloaliphatic hydrocarbon group (for example, a cyclopentyl group, a cyclohexyl group, a cyclopentylmethyl group, a cyclopentylethyl group, a cyclopentylbutyl group, a cyclohexylmethyl group, a cyclohexylethyl group, a cyclohexylbutyl group, a 1 -adamantyl group, a 2-adamantyl group, a 1-adamantylmethyl group, a norbornyl group, a norbornylmethyl group, a tricyclodecanyl group, a tetracyclododecanyl group, a tetracyclododecanylmethyl group, and a dicyclohexylmethyl group); a monovalent unsaturated aliphatic hydrocarbon group (for example, an allyl group and a 3-cyclohexenyl group); an aryl group (for example, a phenyl group, a 1-naphthyl group, and a 2-naphthyl group); an arylalkyl group (for example, a benzyl group and a diphenylmethyl group); and a monovalent hydrocarbon group containing a heteroatom (for example, a tetrahydrofuranyl group, a methoxymethyl group, an ethoxymethyl group, and a methylthiomethyl group, an acetamidemethyl group, a trifluoroethyl group, a (2-methoxyethoxy)methyl group, an acetoxymethyl group, a 2-carboxy-1-cyclohexyl group, a 2-oxopropyl group, a 4-oxo-1-adamantyl group, and a 3-oxocyclohexyl group). Some hydrogens in these groups may be replaced by moieties containing heteroatoms such as oxygen, sulfur, nitrogen, or halogen atoms, or some carbons in these groups may be substituted by moieties containing heteroatoms such as oxygen, sulfur, or nitrogen. Accordingly, these groups may include hydroxy groups, cyano groups, carbonyl groups, carboxyl groups, ether linkages, ester linkages, ester sulfate linkages, carbonates, lactone rings, sultone rings, carboxylic anhydride moieties or haloalkyl moieties.

The term “alkyl group” used herein refers to a linear or branched saturated aliphatic hydrocarbon monovalent group, and examples thereof include a methyl group, an ethyl group, a propyl group, an isobutyl group, a sec-butyl group, a ter-butyl group, a pentyl group, an iso-amyl group, a hexyl group, and the like.

The term “halogenated alkyl group” used herein refers to a group in which one or more substituents of an alkyl group are substituted with halogen, and examples include CF₃ and the like. Here, halogen is F, Cl, Br or I.

The term “alkoxy group” used herein refers to a monovalent group represented by -OA₁₀₁, where A₁₀₁ is an alkyl group. Examples thereof include a methoxy group, an ethoxy group, an isopropyloxy group, and the like.

The term “cycloalkyl group” used herein refers to a monovalent saturated hydrocarbon cyclic group, and examples thereof include monocyclic groups such as a cyclopropyl group, a cyclobutyl group, a cyclopentyl group, a cyclohexyl group, a cycloheptyl group, and the like, and polycyclic condensed cyclic groups such as a norbornyl group and an adamantyl group.

The term “cycloalkoxy group” used herein refers to a monovalent group represented by -OA₁₀₂, where A₁₀₂ is a cycloalkyl group. Examples thereof include a cyclopropoxy group, a cyclobutoxy group, and the like.

The term “aryl group” used herein refers to a monovalent group having a carbocyclic aromatic system, and examples include a phenyl group, a naphthyl group, an anthracenyl group, a phenanthrenyl group, a pyrenyl group, and a chrysenyl group.

Hereinafter, some example embodiments according to the present disclosure will be described in detail with reference to the drawings, and in the description with reference to the drawings, substantially the same or corresponding elements are denoted with the same reference numerals, and overlapping descriptions thereof will be omitted. Regarding the drawings, the thickness is shown enlarged to clearly express the various layers and regions. Also, in the drawings, the thicknesses of some layers and regions are exaggerated for convenience of description. On the other hand, the embodiments described below are merely illustrative, and various modifications can be made on these embodiments.

### [Organic Salt]

An organic salt according to embodiments is represented by Formula 1:

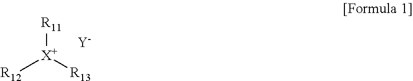

wherein, in Formula 1, X⁺ may be a cation of selenium (Se⁺) or tellurium (Te⁺); R₁₁ to R₁₃ may each independently be a linear, branched, or cyclic C₁-C₂₀ monovalent hydrocarbon group, and at least one of R₁₁ to R₁₃ include at least one iodine (I), and Y⁻ is a counter anion.

For example, X⁺ in Formula 1 may be Te⁺. According to at least some embodiments, the C₁-C₂₀ monovalent hydrocarbon group may optionally include a heteroatom. According to at least some embodiments, two adjacent of R₁₁ to R₁₃ may be bonded to each other to form a ring.

In at least some embodiments, R₁₁ to R₁₃ in Formula 1 may each independently be a C₁-C₂₀ alkyl group, a C₃-C₂₀ cycloalkyl group, or a C₆-C₂₀ aryl group, each unsubstituted or substituted with deuterium, halogen, a cyano group, a hydroxyl group, a carboxyl group, an ester moiety, an ester sulfate moiety, a carbonate moiety, a lactone moiety, a sultone moiety, a carboxylic anhydride moiety, a C₁-C₂₀ alkyl group, a C₁-C₂₀ halogenated alkyl group, a C₁-C₂₀ alkoxy group, a C₃-C₂₀ cycloalkyl group, a C₆-C₂₀ aryl group, and/or a combination thereof; at least one of R₁₁ to R₁₃ includes at least one I; and two adjacent ones of R₁₁ to R₁₃ may be optionally bonded to each other to form a ring.

For example, R₁₁ to R₁₃ in Formula 1 may each independently be at least one of a C₁-C₂₀ alkyl group, a C₃-C₂₀ cycloalkyl group, or a C₆-C₂₀ aryl group, each unsubstituted or substituted with at least one of halogen, a cyano group, a hydroxyl group, an ester moiety, an ester sulfate moiety, a lactone moiety, a sultone moiety, a carboxylic anhydride moiety, a methyl group, an ethyl group, a propyl group, an isopropyl group, a butyl group, an isobutyl group, a sec-butyl group, a tert-butyl group, a halogenated methyl group, a halogenated ethyl group, a methoxy group, an ethoxy group, a phenyl group, and/or a combination thereof, at least one of R₁₁ to R₁₃ includes at least one I, and adjacent two of R₁₁ to R₁₃ may be optionally bonded to each other to form a ring.

In at least some embodiments, at least one of R₁₁ to R₁₃ in Formula 1 may be a C₆-C₂₀ aryl group substituted with at least one I.

In at least some embodiments, one of R₁₁, R₁₂, or R₁₃ includes at least one I; R₁₁ and R₁₂ each include at least one I, and R₁₃ does not include I; and/or each of R₁₁ to R₁₃ may include at least one I.

In at least some embodiments, one of R₁₁, R₁₂, or R₁₃ includes more than one I; R₁₁ and R₁₂ each include more than one I and R₁₃ does not include I; and/or each of R₁₁ to R₁₃ may include more than one I.

In at least some embodiments, the organic salt may include one, two, or three I(s).

In at least some embodiments, the organic salt represented by Formula 1 may be represented by Formula 1-1:

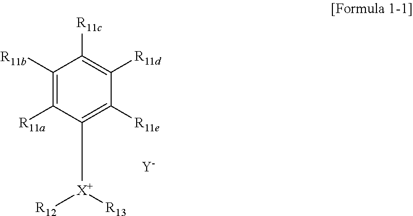

wherein, in Formula 1-1, X⁺ may be Se⁺or Te⁺; R₁₁ₐ to R₁₁ₑ may each independently be hydrogen, halogen, or a linear, branched or cyclic C₁-C₂₀ monovalent hydrocarbon group; R₁₂ and R₁₃ may each independently be a linear, branched, or cyclic C₁-C₂₀ monovalent hydrocarbon group, at least one of R₁₁ₐ to R₁₁ₑ, R₁₂, and R₁₃ may include at least one I, adjacent two of R₁₁ₐ to R₁₁ₑ, R₁₂, and R₁₃ may optionally be bonded to each other to form a ring, and Y⁻ may be a counter anion.

In at least some embodiments at least one of R₁₁ₐ to R₁₁ₑ, R₁₂, and/or R₁₃ which may optionally include a heteroatom. For example, at least one of R₁₁ₐ to R₁₁ₑ in Formula 1-1 may be I. In at least some embodiments, adjacent two of R₁₁ₐ to R₁₁ₑ, R₁₂, and R₁₃ may optionally be bonded to each other to form a ring.

In at least one embodiment, R₁₁ₑ in Formula 1-1 may be I.

In at least some embodiments, the organic salt represented by Formula 1 may be represented by Formula 1-11 or 1-12:

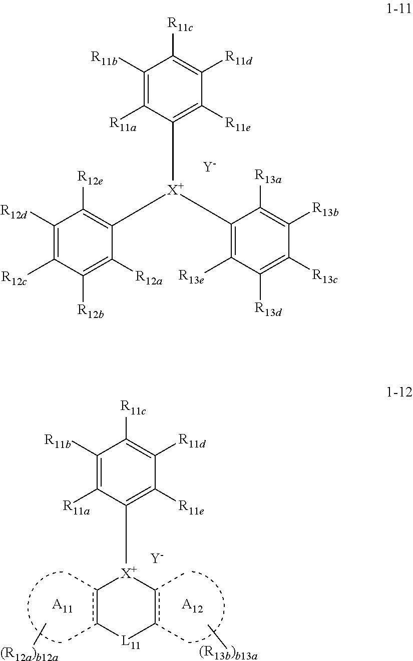

wherein, in Formulae 1-11 and 1-12, X⁺ may be Se⁺ or Te⁺; R₁₁ₐ to R₁₁ₑ, R₁₂ₐ to R₁₂ₑ, and R₁₃ₐ to R₁₃ₑ may each independently be: hydrogen; deuterium, halogen; or a linear, branched, or cyclic C₁-C₂₀ monovalent hydrocarbon group that may optionally include a heteroatom; at least one of R₁₁ₐ to R₁₁ₑ may be I; b12a and b13a may each be an integer from 1 to 4; A₁₁ and A₁₂ may each be absent or a benzene ring; the respective [symbol] may be a carbon-carbon single bond, a resonant pi-bond (e.g., in a condensed ring), and/or a carbon-carbon double bond; L₁₁ may be a single bond, O, S, CO, SO, SO₂, CRR′, or NR; R and R′ may each independently be hydrogen, deuterium, halogen, a hydroxyl group, a C₁-C₂₀ alkyl group, a C₁-C₂₀ halogenated alkyl group, a C₁-C₂₀ alkoxy group, a C₃-C₂₀ cycloalkyl group and/or a C₃-C₂₀ cycloalkoxy group; and Y⁻ may be a counter anion. In at least some embodiments, adjacent two of R₁₁ₐ to R₁₁ₑ, R₁₂ₐ to R₁₂ₑ, and R₁₃ₐ to R₁₃ₑ may optionally be linked to form a condensed ring.

In at least some embodiments, at least one of R₁₁ₐ to R₁₁ₑ in Formulae 1-11 and 1-12 may be I.

In at least some embodiments, R_{11c} in Formulae 1-11 and 1-12 may be I.

In at least some embodiments, A₁₁ and A₁₂ may be absent; A₁₁ may be benzene and A₁₂ may be absent; or A₁₁ and A₁₂ may each simultaneously be benzene.

In at least some embodiments, A₁₁ and A₁₂ may be absent at the same time, or A₁₁ and A₁₂ may both be benzene.

In at least some embodiments, Y⁻ in Formula 1 may be represented by at least one of Formulae 2-1 to 2-4:

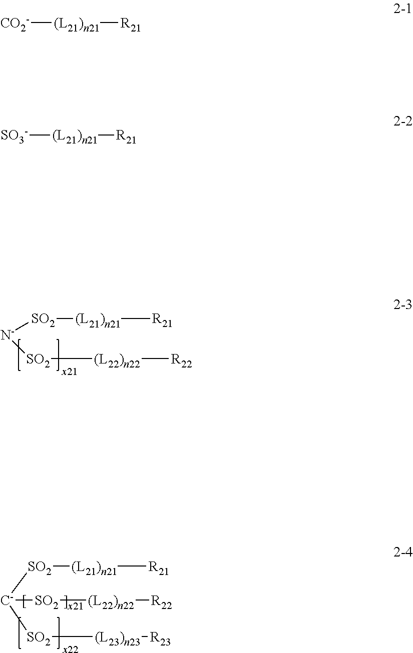

wherein, in Formulae 2-1 to 2-4, L₂₁ to L₂₃ may each independently be a single bond or CRR′, R and R′ may each independently be hydrogen, deuterium, halogen, a hydroxyl group, a C₁-C₂₀ alkyl group, a C₁-C₂₀ halogenated alkyl group, a C₁-C₂₀ alkoxy group, a C₃-C₂₀ cycloalkyl group and/or a C₃-C₂₀ cycloalkoxy group; n21 to n23 may each independently be 1, 2, or 3; x21 and x22 may each independently be 0 or 1; R₂₁ to R₂₃ may each independently be: hydrogen; halogen; or a linear, branched or cyclic C₁-C₂₀ monovalent hydrocarbon group that may optionally include a heteroatom.

For example, R₂₁ to R₂₃ in Formulae 2-1 to 2-4 may each independently be: hydrogen; halogen; or a C₁-C₂₀ alkyl group, a C₃-C₂₀ cycloalkyl group, or a C₆-C₂₀ aryl group, each unsubstituted or substituted with deuterium, halogen, a cyano group, a hydroxyl group, a carboxyl group, an ester moiety, an ester sulfate moiety, a carbonate moiety, a lactone moiety, a sultone moiety, a carboxylic anhydride moiety, a C₁-C₂₀ alkyl group, a C₁-C₂₀ halogenated alkyl group, a C₁-C₂₀ alkoxy group, a C₃-C₂₀ cycloalkyl group, a C₆-C₂₀ aryl group, a C₃-C₂₀ heteroaryl group, and/or a combination thereof.

In at least some example embodiments, Y⁻ in Formula 1 may be represented by at least one of Formulae 2-1 to 2-3.

In at least some example embodiments, Y⁻ in Formula 1 may be represented by at least one of Formulae 2-21 to 2-23:

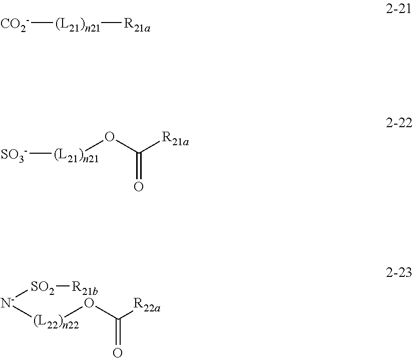

In Formulae 2-21 to 2-23, L₂₁ and L₂₂ may each independently be a single bond or CXₐX_{b}; Xₐ and X_{b} may each independently be hydrogen, halogen, a C₁-C₂₀ alkyl group, or a C₁-C₂₀ halogenated alkyl group; n21 and n22 may each independently be 1, 2, or 3; R₂₁ₐ, R₂₂ₐ, and R_{21b} may each independently be: a C₁-C₂₀ alkyl group; a C₁-C₂₀ halogenated alkyl group; a cyclohexyl group, an adamantyl group, a norbornyl group, a tricyclodecanyl group, a tetracyclododecanyl group, a phenyl group, or a naphthyl group, each unsubstituted or substituted with halogen, a hydroxyl group, a C₁-C₂₀ alkyl group, a C₁-C₂₀ halogenated alkyl group, a C₃-C₂₀ cycloalkyl group, a C₆-C₂₀ aryl group, a C₃-C₂₀ heteroaryl group, and/or a combination thereof.

In at least some embodiments, R₂₂ₐ in Formula 2-23 may be a cyclohexyl group, an adamantyl group, norbornyl, a tricyclodecanyl group, a tetracyclododecanyl group, a phenyl group, or a naphthyl group, each unsubstituted or substituted with halogen, a C₁-C₂₀ alkyl group, a C₁-C₂₀ halogenated alkyl group, a C₃-C₂₀ cycloalkyl group, an C₆-C₂₀ aryl group, a C₃-C₂₀ heteroaryl group, and/or a combination thereof. In at least some embodiments, R_{21b} may be a C₁-C₂₀ alkyl group or a C₁-C₂₀ halogenated alkyl group.

In at least some embodiments, Y⁻ in Formula 1 may be selected from Group A:

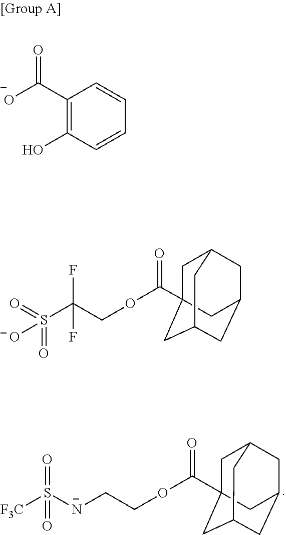 [SMILES: O=C([O-])c1ccccc1O ; O=C(OCC(F)(F)S(=O)(=O)[O-])C12CC3CC(CC(C3)C1)C2 ; O=C(OCC[N-]S(=O)(=O)C(F)(F)F)C12CC3CC(CC(C3)C1)C2]

In at least some embodiments, the organic salt represented by Formula 1 may be selected from Group I:

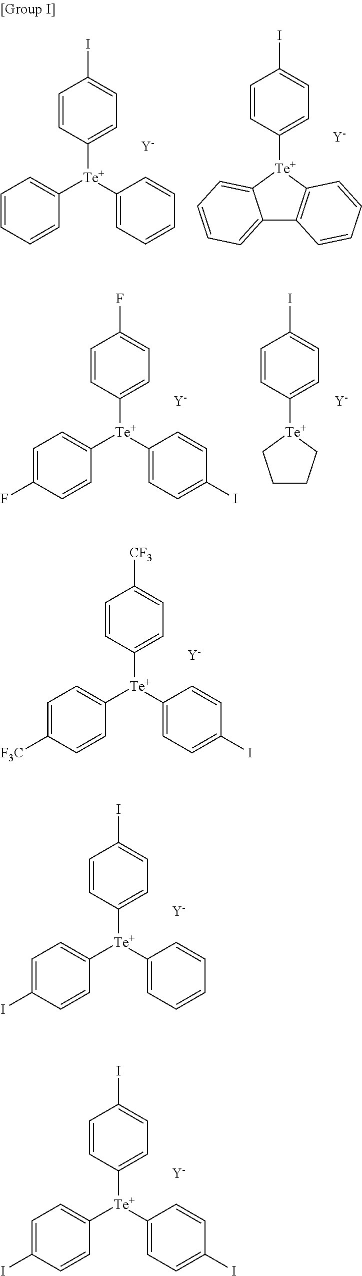

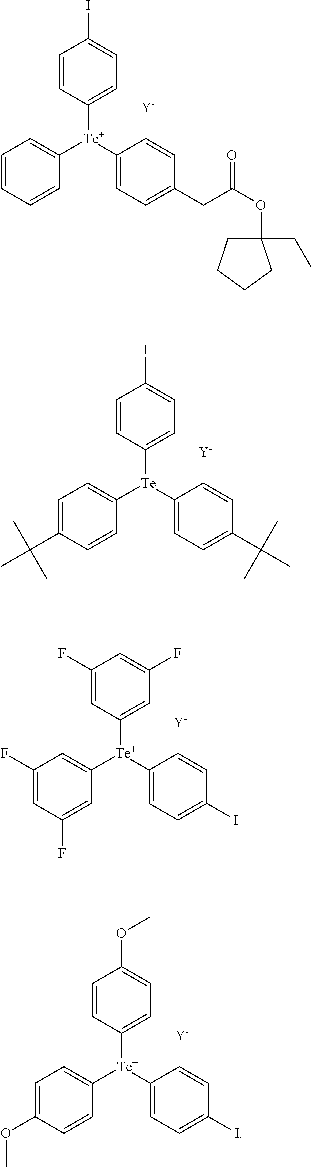

Typically, in EUV (13.5 nm) noise increases significantly at the boundary area between the area exposed by the EUV light source and the unexposed area that is not exposed compared to, e.g., an argon fluoride (ArF) immersion light sources, since EUV has a lower number of photons and the lower the exposure dose is the noisier the boundary area. In the case of a lithography process using an EUV light source, in order to compensate for this, a larger amount of a photoacid generator may be used compared to a lithography process using another light source having the same amount of light. However, when the photoresist composition includes a high content of a photoacid generator, the glass transition temperature (Tg) of the base resin may change and thermal stability thereof may deteriorate. In addition, the resolution of resist patterns formed due to a photoacid generator remaining during a lithography process using an EUV light source, may deteriorate.

In the case of the organic salt represented by Formula 1, X⁺ is not S⁺ but Se⁺ or Te⁺, and thus, the photo sensitivity can be improved compared to an organic salt containing S⁺.

In addition, since iodine (I) has a significantly higher light absorptivity of about 1.4×10⁷ cm²/mol or more compared to hydrogen, carbon, fluorine, chlorine, or bromine, the organic salt represented by Formula 1 containing at least one I may have higher absorptivity.

Therefore, even when a photoresist composition including the organic salt represented by Formula 1 uses a relatively small amount of the organic salt represented by Formula 1, for example, the same or smaller amount thereof than when a light source other than EUV is used, the improved resolution pattern may be obtained when a pattern is formed using an EUV light source.

### [Photoresist Composition]

Another aspect provides a photoresist composition including the organic salt, an organic solvent, and a base resin. The photoresist composition may have, for example, improved developability and/or improved resolution.

The solubility of the photoresist composition in a developing solution is changed by exposure to high energy rays, such as EUV. The photoresist composition may be a positive photoresist composition corresponding to a case where an exposed portion of the photoresist film is dissolved and removed to form a positive photoresist pattern, or a negative photoresist composition corresponding to a case where an unexposed portion of the photoresist film is dissolved and removed to form a negative photoresist pattern. In addition, the photoresist composition according to an embodiment may be used for an alkali developing process using an alkali developing solution for a developing process in forming a photoresist pattern, or may be used for a solvent developing process using a developing solution containing an organic solvent for the developing process (hereinafter referred to as an organic developing solution).

The organic salt may be a photo-decomposable compound that may be decomposed by exposure to light. The organic salt may act as a photoacid generator since an acid is generated when decomposed by exposure to light, and thus, the photoresist composition may not include a separate photoacid generator. Instead, the photoresist composition may further include a quencher. In at least some embodiments, the organic salt may be a photodegradable compound which may generate an acid by exposure to light and, before the exposure, may act as a quenching base to neutralize the acid. In this case, the organic salt may be used in combination with a photoacid generator that generates an acid. In addition, since the organic salt can generate an acid by exposure, the organic salt may lose the quencher function thereof by neutralization with the acid generated by itself, and thus the contrast between the exposed portion and the unexposed portion may be further enhanced.

The organic salt may be used in an amount of 0.1 parts by weight to 40 parts by weight, or 5 parts by weight to 30 parts by weight, based on 100 parts by weight of the base resin. Within these ranges, the photoacid generator and/or quencher may show at appropriate levels of functions, and any performance loss, for example, the formation of foreign particles due to a decrease in sensitivity and/or lack of solubility may be reduced.

Since the organic salt is as described above, hereinafter, an organic solvent, a base resin, and a photoacid generator, which is an optional component, will be described below. In addition, the photoresist composition may include one kind of organic salt represented by Formula 1 or two (or more) different kinds of organic salts represented by Formula 1 in combination.

### <Organic Solvent>

The organic solvent included in the photoresist composition may be a material that is capable of dissolving or dispersing organic salts, base resins, photoacid generators, and optional components contained as needed. One type of the organic solvent may be used, or two or more different types of solvents may be used in combination. In addition, a mixed solvent in which water is mixed with an organic solvent, may be used.

Examples of the organic solvent include alcohol solvents, ether solvents, ketone solvents, amide solvents, ester solvents, sulfoxide solvents, hydrocarbon solvents and the like.

Examples of alcoholic solvents are: monoalcoholic solvents such as methanol, ethanol, n-propanol, isopropanol, 1-methoxy-2-propanol, 1-ethoxy-2-propanol, n-butanol, isobutanol, sec-butanol, tert-butanol, n-pentanol, isopentanol, 2-methylbutanol, sec-pentanol, tert-pentanol, 3-methoxybutanol, 3-methyl-3-methoxybutanol, n-hexanol, 2 -methylpentanol, sec-hexanol, 2-ethylbutanol, sec-heptanol, 3-heptanol, n-octanol, 2-ethylhexanol, sec-octanol, n-nonylalcohol, 2,6- dimethyl-4-heptanol, n-decanol, sec-undecyl alcohol, trimethylnonyl alcohol, sec-tetradecyl alcohol, sec-heptadecyl alcohol, furfuryl alcohol, phenol, cyclohexanol, methylcyclohexane alcohol, 3,3,5-trimethylcyclohexanol, benzyl alcohol, and diacetone alcohol; polyhydric alcohol solvents such as ethylene glycol, 1,2-propylene glycol, 1,3-butylene glycol, 2,4-pentanediol, 2-methyl-2,4-pentanediol, 2,5-hexanediol, 2,4-heptanediol, 2-ethyl-1,3-hexanediol, diethylene glycol, dipropylene glycol, triethylene glycol, and tripropylene glycol; and polyhydric alcohol-containing ether solvents such as ethylene glycol monomethyl ether, ethylene glycol monoethyl ether, ethylene glycol monopropyl ether, ethylene glycol monobutyl ether, ethylene glycol monohexyl ether, ethylene glycol monophenyl ether, ethylene glycol mono-2-ethylbutyl ether, diethylene glycol monomethyl ether, diethylene glycol monoethyl ether, diethylene glycol monopropyl ether, diethylene glycol monobutyl ether, diethylene glycol monohexyl ether, diethylene glycol dimethyl ether, propylene glycol monomethyl ether, propylene glycol dimethyl ether, propylene glycol monoethyl ether, propylene glycol monopropyl ether, propylene glycol monobutyl ether, dipropylene glycol monomethyl ether, dipropylene glycol monoethyl ether, and dipropylene glycol monopropyl ether.

Examples of the ether-based solvent include dialkylether-based solvents such as diethylether, dipropylether, and dibutylether; cyclic ether-based solvents such as tetrahydrofuran and tetrahydropyran; and aromatic ring-containing ether-based solvents such as diphenylether and anisole.

Examples of ketone solvents are chain ketone solvents, such as acetone, methylethylketone, methyl-n-propyl ketone, methyl-n-butyl ketone, methyl-n-pentyl ketone, diethyl ketone, methyl isobutyl ketone, 2-heptanone, ethyl-n-butyl ketone, methyl-n-hexy ketone, diisobutyl ketone, and trimethyl nonanone; cyclic ketone solvents, such as cyclopentanone, cyclohexanone, cycloheptanone, cyclooctanone, and methylcyclohexanone; 2,4-pentandione, acetonyl acetone, and acetphenone.

Examples of the amide solvent are cyclic amide solvents such as N,N′;-dimethylimidazolidinone and N-methyl-2-pyrrolidone; and chain amide solvents such as N-methylformamide, N,N-dimethylformamide, N,N-diethylformamide, acetamide, N-methylacetamide, N,N-dimethylacetamide, and N-methylpropionamide.

Examples of ester solvents are: acetate ester solvents such as methyl acetate, ethyl acetate, n-propyl acetate, isopropyl acetate, n-butyl acetate, isobutyl acetate, sec-butyl acetate, ter-butyl acetate, n-pentyl acetate, isopentyl acetate, sec-pentyl acetate, 3 -methoxybutyl acetate, methylpentyl acetate, 2-ethylbutyl acetate, 2-ethylhexyl acetate, benzyl acetate, cyclohexyl acetate, methylcyclohexyl acetate, and n-nonyl acetate; polyhydric alcohol-containing ether carboxylate solvents such as ethylene glycol monomethyl ether acetate, ethylene glycol monoethyl ether acetate, diethylene glycol monomethyl ether acetate, diethylene glycol monoethyl ether acetate, diethylene glycol mono-n-butyl ether acetate, propylene glycol monomethyl ether acetate, propylene glycol monoethyl ether acetate, propylene glycol monopropyl ether acetate, propylene glycol monobutyl ether acetate, dipropylene glycol monomethyl ether acetate, and dipropylene glycol monoethyl ether acetate; lactone solvents such as y-butyrolactone and 6-valerolactone; carbonate solvents such as dimethyl carbonate, diethyl carbonate, ethylene carbonate, and propylene carbonate; lactate ester solvents such as methyl lactate, ethyl lactate, n-butyl lactate, and n-amyl lactate; glycoldiacetate, methoxytriglycol acetate, ethyl propionate, n-butyl propionate, isoamyl propionate, diethyloxalate, di-n-butyloxalate, methyl acetoacetate, ethyl acetoacetate, diethyl malonate, dimethyl phthalate, and diethyl phthalate.

Examples of the sulfoxide-based solvent include dimethyl sulfoxide and diethyl sulfoxide.

Examples of the hydrocarbon-based solvent include aliphatic hydrocarbon-based solvents such as n-pentane, isopentane, n-hexane, isohexane, n-heptane, isoheptane, 2,2,4-trimethylpentane, n-octane, isooctane, cyclohexane, and methylcyclohexane; and aromatic hydrocarbon-based solvents, such as benzene, toluene, xylene, mesitylene, ethylbenzene, trimethylbenzene, methylethylbenzene, n-propylbenzene, isopropylbenzene, diethylbenzene, isobutylbenzene, triethylbenzene, diisopropylbenzene, and n-amylnaphthalene.

In at least some embodiments, the organic solvent may be selected from alcohol-based solvents, amide-based solvents, ester-based solvents, sulfoxide-based solvents, and/or a combination thereof. In at least some embodiments, the organic solvent may be selected from propylene glycol monomethyl ether, propylene glycol monoethyl ether, propylene glycol monomethyl ether acetate, N-methyl-2-pyrrolidone, N,N-dimethylacetamide, ethyl lactate, dimethyl sulfoxide, and/or a combination thereof.

On the other hand, when an acid labile group in the form of acetal is used, the organic solvent may further include a high-boiling alcohol such as diethylene glycol, propylene glycol, glycerol, 1,4-butanediol, or 1,3-butanediol in order to accelerate the deprotection reaction of acetal.

The organic solvent may be used in an amount of 200 parts by weight to 5,000 parts by weight, for example, 400 to 3,000 parts by weight, based on 100 parts by weight of the base resin.

### <Base Resin>

The base resin may include a repeating unit containing an acid labile group represented by Formula 4:

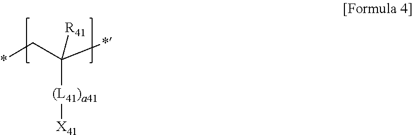

wherein, in Formula 4, R₄₁ may be hydrogen, deuterium, halogen, a C₁-C₂₀ linear or branched alkyl group, or a C₁-C₂₀ linear or branched halogenated alkyl group; L₄₁ may be a single bond, a substituted or unsubstituted C₁-C₁₀ alkylene group, a substituted or unsubstituted C₃-C₁₀ cycloalkylene group, a substituted or unsubstituted C₃-C₁₀ heterocycloalkylene group, a substituted or unsubstituted phenylene group, a substituted or unsubstituted naphthylene group, *—O —*′, *—C(═O)O—*′, —OC(═O)—*′, *—C(═O)NH—*′, —NHC(═O)—*′, and/or a combination thereof, a41 may be an integer selected from 1 to 6; X₄₁ may be an acid labile group, and * and *′ may each be a binding site to a neighboring atom.

For example, R₄₁ in Formula 4 may be hydrogen, deuterium, halogen, CH₃, CH₂F, CHF₂, or CF₃.

The “C₁-C₁₀ alkylene group” of L₄₁ in Formula 4 may be, for example, a methylene group, an ethylene group, a propylene group, a butylene group, or an isobutylene group.

The “C₃-C₁₀ cycloalkylene group” of L₄₁ in Formula 4 may be, for example, a cyclopentylene group, a cyclohexylene group, an adamantylene group, an adamantylmethylene group, a norbornylene group, a norbornylmethylene group, a tricyclodecaneylene group, a tetracyclododecanylene group, a tetracyclododecanylmethylene group, or a dicyclohexylmethylene group.

The “C₁-C₁₀ heterocycloalkylene group” of L₄₁ in Formula 4 may be formed by replacing some of the carbon in the “C₃-C₁₀ cycloalkylene group” by moieties containing heteroatoms such as oxygen, sulfur, or nitrogen. Accordingly, the “C₁-C₁₀ heterocycloalkylene group” may include an ether linkage, an ester linkage, an ester sulfate linkage, carbonate, a lactone ring, a sultone ring, and/or a carboxylic anhydride moiety.

a41 in Formula 4 refers to the number of repetitions of L₄₁, and when a41 is 2 or more, a plurality of L₄₁ may be identical to or different from each other.

In at least some embodiments, X₄₁ in Formula 4 may be represented by at least one of

Formulae 6-1 to 6-7:

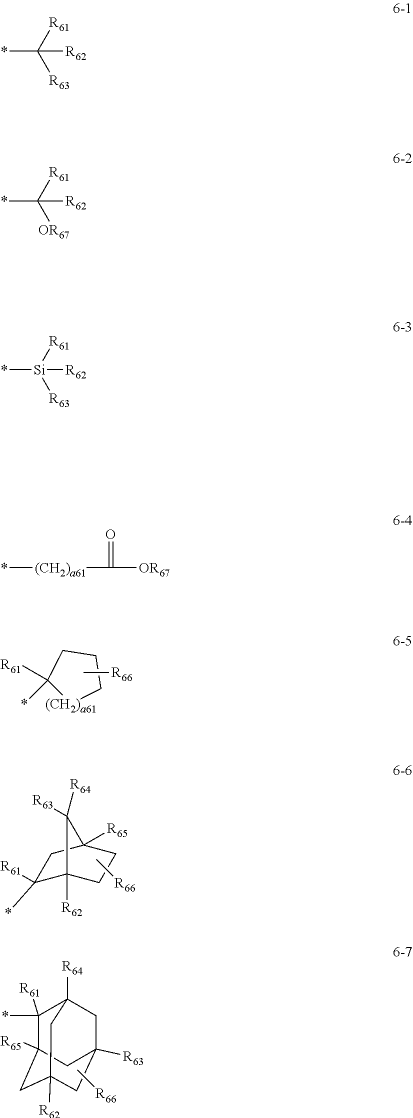

wherein, in Formulae 6-1 to 6-7, a61 may be an integer selected from 0 to 6; R₆₁ to R₆₆ may each independently be: hydrogen; deuterium; or a linear, branched or cyclic C₁-C₂₀ monovalent hydrocarbon group that may optionally include a heteroatom.

R₆₇ may be a linear, branched, or cyclic C₁-C₂₀ monovalent hydrocarbon group which may optionally include a heteroatom; neighboring two groups selected from R₆₁ to R₆₇ may be optionally combined with each other to form a ring, and * is a binding site with a neighboring atom.

When a61 in Formulae 6-4 and 6-5 is 0, (CH₂)ₐ₆₁ may be a single bond.

The “monovalent hydrocarbon group” of R₆₁ to R₆₇ in Formulae 6-1 to 6-7 may be understood by referring to the “monovalent hydrocarbon group” in the list of R₁₁ in Formula 1.

In at least some embodiments, the repeating unit represented by Formula 4 may be represented by one of Formulae 4-1 and 4-2:

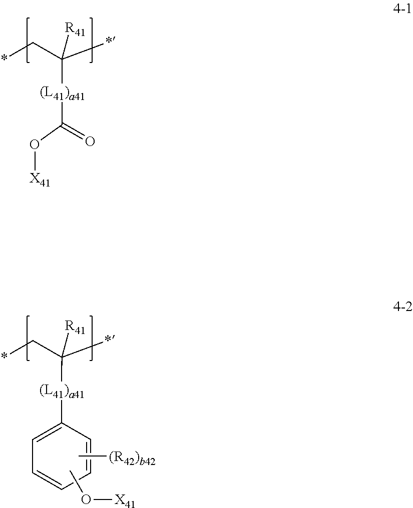

wherein, in Formulae 4-1 and 4-2, the definitions of L₄₁ and X₄₁ are the same as in Formula 4; a41 may be an integer selected from 1 to 4; R₄₂ may be hydrogen, or a linear, branched, or cyclic monovalent hydrocarbon group of C₁-C₁₀ that may optionally include a heteroatom; b42 may be an integer selected from 1 to 4; and * and *′ may each be a binding site to a neighboring atom.

The “monovalent hydrocarbon group” of R₆₁ to R₆₇ in Formula 4-2 may be understood by referring to the “monovalent hydrocarbon group” in the list of R₁₁ in Formula 1.

The base resin containing the repeating unit represented by Formula 4 is decomposed under the action of an acid to generate a carboxyl group, thereby converting to have alkali-solubility.

In addition to the repeating unit represented by Formula 4, the base resin may further include a repeating unit represented by Formula 5:

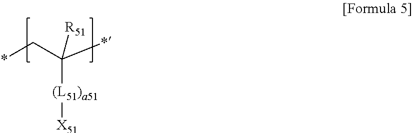

wherein, in Formula 5, R₅₁ may be hydrogen, deuterium, halogen, a C₁-C₂₀ linear or branched alkyl group, or a C₁-C₂₀ linear or branched halogenated alkyl group; L₅₁ may be a single bond, a substituted or unsubstituted C₁-C₁₀ alkylene group, a substituted or unsubstituted C₃-C₁₀ cycloalkylene group, a substituted or unsubstituted C₃-C₁₀ heterocycloalkylene group, a substituted or unsubstituted phenylene group, a substituted or unsubstituted naphthylene group, *—O—*′, *—C(═O)O—*′, —OC(═O)—*′, *—C(═O)NH—*′, —NHC(═O)—*′, or a combination thereof; a51 may be an integer selected from 1 to 6; X₅₁ may be a non-acid labile group, and * and *′ may each be a binding site to a neighboring atom.

For example, R₅₁ in Formula 5 may be understood with reference to the description provided in connection with R₄₁ in Formula 4.

For example, L₅₁ in Formula 5 may be understood with reference to the description provided in connection with L₄₁ in Formula 4.

a51 in Formula 5 refers to the number of repetitions of L₅₁, and when a51 is 2 or more, a plurality of L₅₁ may be identical to or different from each other.

In at least some embodiments, X₅₁ in Formula 5 may be hydrogen, deuterium, or a linear, branched or cyclic C₁-C₂₀ monovalent hydrocarbon group containing at least one polar moiety selected from a hydroxyl group, a halogen, a cyano group, a carbonyl group, a carboxyl group, *—O—*′, *—C(═O)O—*′, —OC(═O)—*′, *—S(═O)O—*′, —OS(═O)—*′, lactone rings, sultone rings, and carboxylic anhydride moieties. Here, the “monovalent hydrocarbon group” can be understood by referring to the “monovalent hydrocarbon group” in the list of R₁₁ of Formula 1, and may necessarily include at least one polar moiety selected from a hydroxyl group, a halogen, a cyano group, a carbonyl group, a carboxyl group, *—O—*′, *—C(═O)O—*′, —OC(═O)—*′, *—S(═O)O—*′, —OS(═O)—*′, lactone rings, sultone rings, and carboxylic anhydride moieties.

In at least some embodiments, the repeating unit represented by Formula 5 may be represented by at least one of Formulae 5-1 and 5-2:

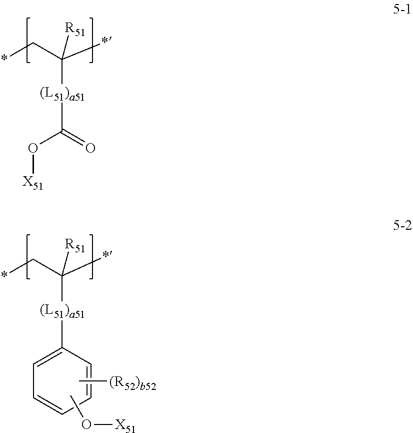

wherein, in Formulae 5-1 and 5-2, the definitions of L₅₁ and X₅₁ are the same as in Formula 5, respectively; a51 may be an integer selected from 1 to 4; R₅₂ may be hydrogen, a hydroxyl group, or a linear, branched or cyclic monovalent C₁-C₁₀ hydrocarbon group that may optionally include a heteroatom;b52 may be an integer selected from 1 to 4; and * and *′ may each be a binding site to a neighboring atom.

The “monovalent hydrocarbon group” of R₅₂ in Formula 5-2 may be understood by referring to the “monovalent hydrocarbon group” in the list of R₁₁ in Formula 1.

For example, in ArF lithography processes, X₅₁ may include a lactone ring as a polar moiety, and in krypton fluoride (KrF), electron beam (EB) and EUV lithography processes, X₅₁ may be a phenol.

In at least some embodiments, the base resin may further include a moiety containing an anion and/or a cation. For example, the base resin may further include a moiety in which a photoacid generator and/or a quencher are induced to bind to the side chain.

The base resin may have a weight average molecular weight (Mw) of 1,000 to 500,000, for example, 3,000 to 100,000, in which the weight average molecular weight (Mw) was measured by gel permeation chromatography using a tetrahydrofuran solvent and polystyrene which is used as a standard material.

The polydispersity index (PDI: Mw/Mn) of the base resin may be 1.0 to 3.0, or 1.0 to 2.0. Within these ranges, the possibility of foreign matter remaining on the pattern may be lowered, or deterioration of the pattern profile may be minimized. Accordingly, the photoresist composition may be more suitable for forming fine patterns.

The base resin may be prepared by any appropriate method, for example, a method in which unsaturated bond-containing monomer(s) is dissolved in an organic solvent and then thermally polymerized in the presence of a radical initiator.

The mole fraction (mol %) of each repeating unit derived from each monomer in the base resin is as follows, but is not limited thereto: i) 1 mol % to 60 mol %, or 5 mol % to 50 mol %, or 10 mol % to 50 mol % of the repeating unit represented by Formula 4; and ii) 40 mol % to 99 mol %, or 50 mol % to 95 mol %, or 50 mol % to 90 mol % of the repeating unit represented by Formula 5.

The base resin may be a single polymer or may include a mixture of two or more polymers which are different in terms of composition, weight average molecular weight, and/or polydispersity index.

### <Photoacid Generator>

The organic salt may act as a photoacid generator since an acid is generated when decomposed by exposure to light, and thus, the photoresist composition may not include a separate photoacid generator.

However, when the organic salt acts as a quencher, the organic salt may be used in combination with a photoacid generator that generates an acid.

The photoacid generator may be a compound configured to generate an acid when exposed to high energy rays such as UV, deep UV (DUV), EB, EUV, X-rays, α-rays, and γ-rays.

The photoacid generator may include a sulfonium salt, an iodonium salt, and/or a combination thereof.

In at least some embodiments, the photoacid generator may be represented by Formula 7:

B₇₁⁺A₇₁⁻ Formula 7

wherein, in Formula 7, B₇₁⁺ may be represented by Formula 7A, and A₇₁⁻ may be represented by one of Formulae 7B to 7D, and B₇₁⁺ and A₇₁⁻ may optionally be linked via a carbon-carbon covalent bond:

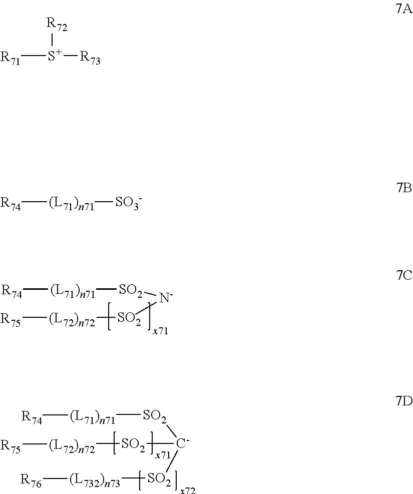

wherein, in Formulae 7A to 7D, L₇₁ to L₇₃ may each independently be a single bond or CRR′;

R and R′ may each independently be hydrogen, deuterium, halogen, a hydroxyl group, a C₁-C₂₀ alkyl group, a C₁-C₂₀ halogenated alkyl group, a C₁-C₂₀ alkoxy group, a C₃-C₂₀ cycloalkyl group or a C₃-C₂₀ cycloalkoxy group; n71 to n73 may each independently be 1, 2, or 3; x71 and x72 may each independently be 0 or 1; R₇₁ to R₇₃ may each independently be a linear, branched or cyclic C₁-C₂₀ monovalent hydrocarbon group; adjacent two of R₇₁ to R₇₃ may be optionally combined with each other to form a ring, and R₇₄ to R₇₆ may each independently be: hydrogen; halogen; or a linear, branched or cyclic C₁-C₂₀ monovalent hydrocarbon group which may optionally include a heteroatom.

For example, in Formula 7, B₇₁⁺ may be represented by Formula 7A, and A₇₁⁻ may be represented by Formula 7B. For example, R₇₁ to R₇₃ in Formula 7A may each be a phenyl group, and R₇₄ in Formula 7B may be a propyl group substituted with F.

The photoacid generator may be included in an amount of 0 parts by weight to 40 parts by weight, 0.1 parts by weight to 40 parts by weight, or 0.1 parts by weight to 20 parts by weight, based on 100 parts by weight of the base resin. Within these ranges, an appropriate level of resolution may be achieved, and problems related to foreign material particles after development or during stripping may be reduced.

One type of the photoacid generator may be used, or two or more different types thereof may be used in combination.

### <Quencher>

The quencher may be a salt that generates an acid having a weaker acidity than the acid generated from the organic salt represented by Formula 1 and/or the photoacid generator.

The quencher may include a sulfonium salt, an iodonium salt, and/or a combination thereof. In at least some embodiments, the quencher may be represented by Formula 8:

B₈₁⁺A₈₁⁻ Formula 8

wherein, in Formula 8, B₈₁⁺ may be represented by any one of Formulae 8A to 8C, and A₈₁⁻ may be represented by any one of Formulae 8D to 8F, and B₈₁⁺ and A₈₁⁻ may optionally be linked via a carbon-carbon covalent bond,

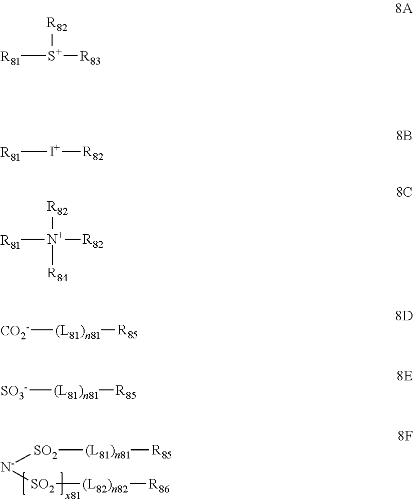

wherein, in Formulae 8A to 8F, R₈₁ to R₈₄ may each independently be a linear, branched or cyclic C₁-C₂₀ monovalent hydrocarbon group; neighboring two groups selected from R₈₁ to R₈₄ may be optionally combined with each other to form a ring; L₈₁ and L₈₂ may each independently be a single bond or CRR′; R and R′ may each independently be hydrogen, deuterium, halogen, a hydroxyl group, a C₁-C₂₀ alkyl group, a C₁-C₂₀ halogenated alkyl group, a C₁-C₂₀ alkoxy group, a C₃-C₂₀ cycloalkyl group or a C₃-C₂₀ cycloalkoxy group; n81 and n82 may each independently be 1, 2, or 3; x81 may be 0 or 1; and R₈₅ and R₈₆ may each independently be: hydrogen; halogen; or a linear, branched or cyclic C₁-C₂₀ monovalent hydrocarbon group which may optionally include a heteroatom.

The quencher may be included in an amount of 0.01 parts by weight to 10 parts by weight, 0.05 parts by weight to 5 parts by weight, or 0.1 parts by weight to 3 parts by weight, based on 100 parts by weight of the base resin. Within these ranges, an appropriate level of resolution may be achieved, and problems related to foreign material particles after development or during stripping may be reduced.

One type of the quencher may be used, or two or more different types thereof may be used in combination.

### <Additional Components>

The photoresist composition may further include at least one of a surfactant, a crosslinking agent, a leveling agent, a colorant, and/or any combination thereof, if needed.

The photoresist composition may further include a surfactant to improve a coating property and developability. Examples of the surfactant include nonionic surfactants such as polyoxyethylene lauryl ether, polyoxyethylene stearyl ether, polyoxyethyleneoleyl ether, polyoxyethylene n-octylphenyl ether, polyoxyethylene n-nonylphenyl ether, polyethyleneglycol dilaurate, and polyethyleneglycol distearate. The surfactant may be a commercially available product in the art or a synthetic product. Examples of the commercially available product of the surfactant are: KP341 (the products of Shin-Etsu Chemical Co., Ltd.); Polyflow No.75 and Polyflow No.95 (the products of Kyoeisha Chemical Co., Ltd.); Ftop EF301, Ftop EF303, and Ftop EF352 (manufactured by Mitsubishi Material Electron Chemical Co., Ltd.); MEGAFACE (registered trademark) F171, MEGAFACE F173, R_{40,} R_{41,} and R₄₃ (the products of DIC Corporation); Fluorad (registered trademark) FC₄₃₀ and Fluorad FC₄₃₁ (the products of 3M Co, Ltd.); AsahiGuard AG710 (the product of AGC Corporation); Surflon (registered trademark) S-382, Surflon SC-101, Surflon SC-102, Surflon SC-103, Surflon SC-104, Surflon SC-105, and Surflon SC-106 (the products of AGC Seimi Chemical Co., Ltd.).

The surfactant may be included in an amount of 0 parts by weight to 20 parts by weight based on 100 parts by weight of the base resin. One type of the surfactant may be used, or two or more different types thereof may be used in combination.

The method for preparing the photoresist composition is not particularly limited. For example, an organic salt, a base resin, a photoacid generator, and any component that is optionally added if needed, may be mixed in an organic solvent. The temperature or time during mixing is not particularly limited. If necessary, filtration may be performed after mixing.

### [Pattern Formation Method]

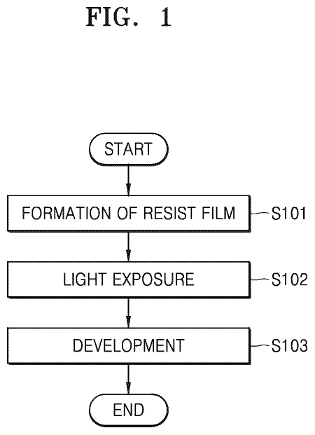

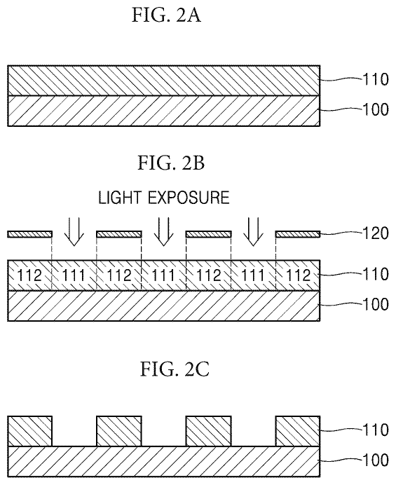

Hereinafter, a pattern formation method according to some embodiments will be described in more detail with reference to FIGS. 1 and 2. FIG. 1 is a flowchart illustrating a pattern formation method according to embodiments, and FIGS. 2A, 2B and 2C are side cross-sectional views used to describe a pattern formation method according to embodiments. Hereinafter, an example of a pattern formation method using a positive photoresist composition will be described, and embodiments of the present disclosure are not limited thereto.

Referring to FIG. 1, the pattern formation method may include forming a photoresist film by applying a photoresist composition (S101), exposing at least a portion of the photoresist film with high energy rays (S102), and forming a photoresist film by developing the photoresist film using a developing solution (S103). The example illustrated in the figures is based on a positive photoresist pattern. In at least some examples, the steps may be omitted if necessary, and/or may be performed in a different order, and/or additional steps may be added.

First, a substrate 100 is prepared. The substrate 100 may be, for example, a semiconductor substrate, such as a silicon substrate or a germanium substrate, and/or may be formed using glass, quartz, ceramic, copper, and/or the like. In some embodiments, the substrate 100 may include a Group III-V compound such as GaP, GaAs, GaSb, and/or the like.

A photoresist film 110 may be formed by applying a photoresist composition to a desired thickness on the substrate 100, e.g., by a coating method. If needed, heating may be performed to remove the organic solvent remaining in the photoresist film 110. As the coating method, spin coating, dipping, roller coating. Or other general coating methods may be used. In at least some embodiments, spin coating may be used, and the thickness of the photoresist film 110 may be adjusted by controlling the viscosity, concentration, and/or spin speed of the photoresist composition. In an embodiment, the thickness of the photoresist film 110 may be from 10 nm to 300 nm. In at least some embodiments, the thickness of the photoresist film 110 may be from 30 nm to 200 nm.

The lower limit of the prebaking temperature may be 60° C. or higher, for example, 80° C. or higher. In addition, the upper limit of the prebaking temperature may be 150° C. or less, specifically 140° C. or less. For example, the prebaking may be performed at a temperature between 60° C. to 150° C. The lower limit of the prebaking time may be 5 seconds or more, for example, 10 seconds or more. The upper limit of the prebaking time may be 600 seconds or less, for example, 300 seconds or less.

Before coating the photoresist composition on the substrate 100, an etch target film (not shown) may be further formed on the substrate 100. The etch target film may refer to a layer on which an image is transferred from a photoresist pattern and converted into a certain pattern. In an embodiment, the etch target film may include an insulating material such as silicon oxide, silicon nitride, or silicon oxynitride. In some embodiments, the etch target film may be formed to include a conductive material such as metal, metal nitride, metal silicide, or metal silicide nitride. In some embodiments, the etch target film may be formed to include a semiconductor material such as polysilicon.

In some embodiments, an anti-reflection film may be further formed on the substrate 100 to maximize the efficiency of the photoresist. The anti-reflection film may be an organic or inorganic anti-reflection film.

In some embodiments, a protective film may be further provided on the photoresist film 110 in order to reduce the influence of alkaline impurities included in the process. Further, in the case of immersion exposure, for example, a protective film for immersion may be provided on the photoresist film 110 to avoid direct contact between the immersion medium and the photoresist film 110.

Next, at least a portion of the photoresist film 110 may be exposed to high energy rays. For example, high energy rays passing through a mask 120 may be irradiated to at least a portion of the photoresist film 110. As such, the photoresist film 110 may have an exposed portion 111 and a non-exposed portion 112.

In some cases, this exposure is performed by irradiating high-energy rays through a mask having a certain pattern using a liquid such as water as a medium. Examples of the high-energy rays include electromagnetic waves such as ultraviolet rays, deep ultraviolet rays, extreme-ultraviolet rays (EUV, wavelength 13.5 nm), X-rays, and γ-rays; and a charged particle beams, such as an electron beam (EB) and an alpha ray. Irradiation of these high-energy rays may be collectively referred to as “exposure” or “exposure to light” herein.

As the exposure light source, the emission of the far ultraviolet region of laser light, such as KrF excimer laser (wavelength 248 nm), ArF excimer laser (wavelength 193 nm), or F₂ excimer laser (wavelength 157 nm), the emission of harmonic laser light in the far ultraviolet region or vacuum ultraviolet region by converting the wavelength of laser light from a solid-state laser light source (YAG or semiconductor laser, etc.), and irradiation of electron beams or extreme ultraviolet (EUV), etc. may be used. During exposure, exposure is usually performed through a mask corresponding to a desired pattern, but when the exposure light source is an electron beam (EB), exposure may be performed by direct drawing without using a mask.

The integrated dose of high-energy rays, for example, ultra-ultraviolet rays, may be 2000 mJ/cm² or less, for example, 500 mJ/cm² or less. In addition, when an electron beam is used as the high-energy ray, the integrated dose of rays may be 5000 μC/cm² or less, for example, 1000 μC/cm² or less.

In addition, post exposure bake (PEB) may be performed after exposure. The lower limit of the temperature of PEB may be 50° C. or higher, for example, 80° C. or higher. The upper limit of the temperature of PEB may be 180° C. or less, for example, 130° C. or less. For example, the PEB may be performed at a temperature between 50° C. to 130° C. The lower limit of the PEB time may be 5 seconds or more, for example, 10 seconds or more. The upper limit of the PEB time may be 600 seconds or less, for example, 300 seconds or less.

Next, the exposed photoresist film 110 may be developed using a developing solution. The exposed portion 111 may be washed away by using the developing solution, and the non-exposed portion 112 remains unwashed away by the developing solution.

Examples of the developing solution include an alkaline developing solution and a developing solution containing an organic solvent (hereinafter, also referred to as “organic developing solution”). Examples of the developing method include a dipping method, a puddle method, a spray method, and a dynamic dosing method. The development temperature may be, for example, 5° C. or more and 60° C. or less, and the developing time may be, for example, 5 seconds or more and 300 seconds or less.

An example of the alkaline developing solution is an alkaline aqueous solution in which one or more kinds of alkaline compounds, such as sodium hydroxide, potassium hydroxide, sodium carbonate, sodium silicate, sodium metasilicate, aqueous ammonia, ethylamine, n-propylamine, diethylamine, di-n-propylamine, triethylamine, methyldiethylamine, ethyldimethylamine, triethanolamine, tetramethyl ammonium hydroxide (TMAH), pyrrole, piperidine, choline, 1,8-diazabicyclo[5.4.0]-7-undecene (DBU), and 1,5-diazabicyclo[4.3.0]-5-nonene (DBN) are dissolved. The alkaline developing solution may further include a surfactant.

The lower limit of the alkaline compound content in the alkaline developing solution may be 0.1 mass % or more, or 0.5 mass % or more, or 1 mass %. In addition, the upper limit of the alkaline compound content in the alkaline developing solution may be 20 mass % or less, or 10 mass % or less, or 5 mass % or less.

After development, the photoresist pattern may be washed with ultrapure water, and then water remaining on the substrate and the pattern may be removed.

As the organic solvent included in the organic developing solution, for example, the same organic solvent as exemplified in the <organic solvent> section of the [Resist Composition] may be used.

The lower limit of the organic solvent content in the organic developing solution may be 80 mass % or more, 90 mass % or more, 95 mass % or more, or 99 mass % or more.

The organic developing solution may include a surfactant. In at least some embodiments, organic developing solutions may include a trace amount of moisture. In addition, at the time of development, development may also be stopped by substituting with a solvent of a different kind from the organic developing solution.

The photoresist pattern after development may be further cleaned. Ultrapure water, rinsing liquid, etc. may be used as a cleaning liquid. The rinsing liquid is not particularly limited as long as it does not dissolve the photoresist pattern, and a solution containing a general organic solvent may be used. For example, the rinsing liquid may be an alcohol-based solvent or an ester-based solvent. After cleaning, the rinsing liquid remaining on the substrate and pattern may be removed. In addition, when ultrapure water is used, water remaining on the substrate and the pattern may be removed.

In addition, the developing solution may be one type or two or more types in combination.

After the photoresist pattern is formed as described above, a patterned interconnection substrate may be obtained. Etching may be performed by known methods such as dry etching using a plasma gas and/or wet etching using an alkaline solution, copper(II) chloride solution, ferric(III) chloride solution, and/or the like.

After the resist pattern is formed, plating may be performed. The plating method is not particularly limited, and examples thereof include copper plating, solder plating, nickel plating, gold plating, and/or the like.

The photoresist pattern remaining after etching may be peeled with an organic solvent. Examples of such an organic solvent include, but are not particularly limited to, propylene glycol monomethyl ether acetate (PGMEA), propylene glycol monomethyl ether (PGME), ethyl lactate (EL), and/or the like. The peeling method is not limited and may be, for example, the immersion method or the spray method. In addition, the interconnection substrate on which the photoresist pattern is formed, may be a multi-layer interconnection substrate or may have small-diameter through-holes.

In at least some embodiments, the interconnection substrate may be formed by a lift-off method in which a photoresist pattern is formed and then metal is deposited in a vacuum and then the photoresist pattern is dissolved using a solution.

The present disclosure will be described in more detail using Examples and Comparative Examples, but the technical scope of the present disclosure is not limited to the following Examples.

### EXAMPLES

### Synthesis Example 1: Synthesis of Compound A

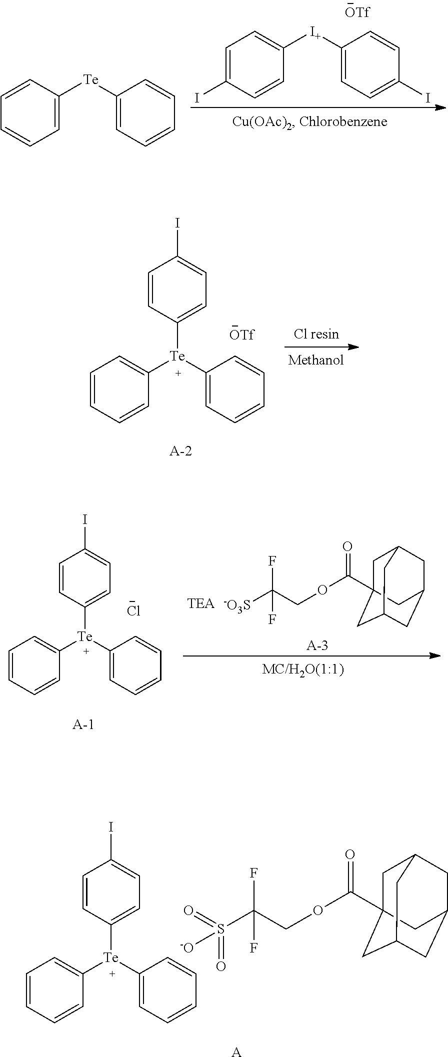

### Synthesis of Compound A-2

Diphenyltellane (3 g, 10.65 mmol), bis(4-iodophenyl)iodonium trifluoromethanesulfonate (7.99 g, 11.71 mmol), and copper acetate (Cu(Oac)₂)(0.164 g, 1.06 mmol) were dissolved in 30 ml of chlorobenzene, and then, stirred at a temperature of 120° C. for 12 hours. Thereafter, the reaction solvent was distilled off under reduced pressure, and the obtained residue was separated and purified by silica gel column chromatography to obtain Compound A-2 (1.05 g, 15.6%). The resulting compound was confirmed by ¹H-NMR.

¹H-NMR (500 MHz, CD₂Cl₂): δ 7.92(m, 2H), 7.68(m, 2H), 7.62-7.59(m, 8H), 7.34(d, 2H)

### Synthesis of Compound A-1

Compound A-2 (1 g, 1.58 mmol) and Cl resin (3 g) were mixed with 10 mL of methanol, and then, stirred for 2 hours. Thereafter, the filtrate obtained by filtration was distilled off under reduced pressure to obtain Compound A-1 (0.75 g, 92%). The resulting compound was confirmed by ¹H-NMR.

¹H-NMR (500 MHz, CD₂Cl₂): δ 7.74(m, 6H), 7.5(m, 4H), 7.44(m, 4H)

### Synthesis of Compound A-3

1.24 g of sodium 1,1-difluoro-2-hydroxyethane-1-sulfonate was added to 20 ml of dichloromethane. 0.51 g of trimethylamine and 0.0641 g of 4-dimethylaminopyridine (DMAP) were added to 10 ml of dichloromethane, and then the mixed solution was slowly added dropwise to the previously prepared solution. 1 g of (3r,5r,7r)-adamantane-1-carbonyl chloride was dissolved in 20 ml of dichloromethane, and then, slowly added dropwise to the mixture at 0° C. After reacting for 16 hours, extraction was performed with dichloromethane and H₂O, and then, the organic layer was collected and dried by adding sodium sulfate (Na₂SO₄). The solvent was removed from the obtained organic layer, thereby obtaining 0.95 g of triethylammonium 2-(1-adamantanecarbonyloxy)-1,1-difluoroethanesulfonate. The resulting compound was confirmed by ¹H-NMR.

¹H-NMR (500 MHz, CD₂Cl₂): δ 4.64(t, 2H), 3.13(m, 6H), 2.0(s, 3H), 1.92(t, 6H), 1.73(t, 6H), 1.34(m, 9H).

### Synthesis of Compound A

Compound A-1 (0.7 g, 1.35 mmol) and triethylamine, 2-((adamantane-1-carbonyl)oxy)-1,1-difluoroethane-1-sulfonate salt (0.6 g, 1.41 mmol) were mixed with 5 mL of methylene chloride (DCM) and 10 mL of distilled water (DW), and then, stirred for 2 hours. Thereafter, the organic layer was separated, dried using MgSO₄, and filtered, the filtrate obtained therefrom was removed, and the obtained residue was separated and purified by silica gel column chromatography to obtain Compound A (0.8 g, 73.6%). The resulting compound was confirmed by ¹H-NMR and LC-MS.

¹H-NMR (500 MHz, CD₂Cl₂): δ 7.9(d, 2H), 7.7-7.5(m, 10H), 7.34(d, 2H), 4.49(t, 2H), 1.97(s, 3H), 1.87(s, 6H), 1.7(m, 6H), LC-MS m/z=486.9(cation).

### Synthesis Example 2: Synthesis of Compound B

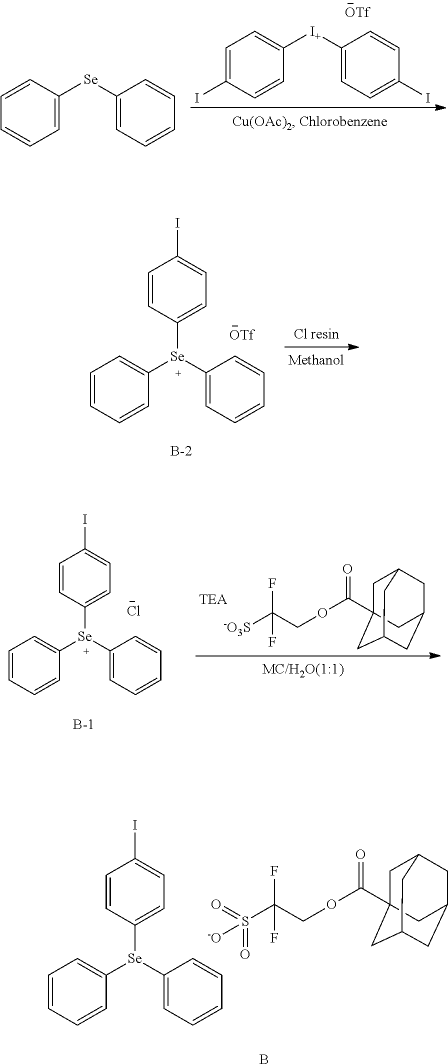

### Synthesis of Compound B-2

Diphenylselane (1 g, 4.29 mmol) (Sigma-Aldrich reagent), bis(4-iodophenyl)iodonium trifluoromethanesulfonate (3.22 g, 4.72 mmol), and copper acetate (Cu(Oac)₂) (0.78 g, 0.43 mmol) were dissolved in 30 ml of chlorobenzene and stirred at 120° C. for 12 hours. Thereafter, the reaction solvent was distilled off under reduced pressure, and the obtained residue was separated and purified by silica gel column chromatography to obtain Compound B-2 (1.3 g, 51.8%). The resulting compound was confirmed by ¹H-NMR.

¹H-NMR (500 MHz, CD₂Cl₂): δ 8.03(m, 2H), 7.79(m, 2H), 7.69(m, 4H), 7.63(m, 4H), 7.34(d, 2H).

### Synthesis of Compound B-1

Compound B-1 was obtained in the same manner as used to synthesize Compound A-1, except that Compound B-2 was used instead of Compound A-2. The resulting compound was confirmed by ¹H-NMR.

¹H-NMR (500 MHz, CD₂Cl₂): δ 7.92(d, 2H), 7.72-7.74(m, 4H), 7.68(m, 2H), 7.60(m, 4H), 7.5(m, 2H).

### Synthesis of Compound B

Compound B was obtained in the same manner as used to synthesize Compound A of Synthesis Example 1, except that Compound B-1 was used instead of Compound A-1. The resulting compound was confirmed by ¹H-NMR and LC-MS.

¹H-NMR (500 MHz, CD₂Cl₂): δ 8.0(d, 2H), 7.74(m, 2H), 7.66(m, 4H), 7.64(m, 4H), 7.34(m, 2H), 4.58(t, 2H), 1.99(s, 3H), 1.89(s, 6H), 1.7(m, 6H), LC-MS m/z=436.92(cation).

### Synthesis Example 3: Synthesis of Compound C

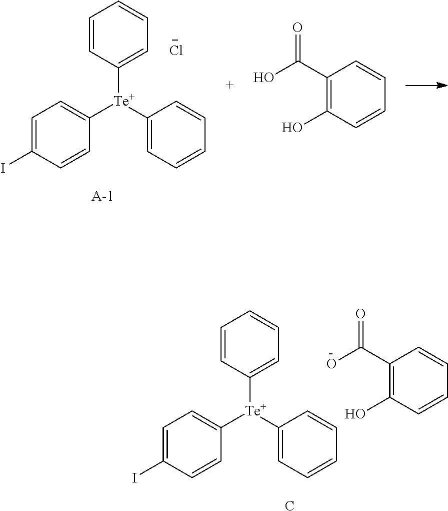

Compound A-1 (0.4 g, 0.77 mmol) and salicylic acid (Sigma-Aldrich) (0.11 g, 0.81 mmol) were mixed with 10 ml of dichloromethane and 10 ml of 1N NaOH aqueous solution, followed by stirring for 2 hours. Thereafter, the organic layer was separated, dried using MgSO₄, and filtered, the solvent was removed under reduced pressure, and the resulting solid was washed with ether to obtain Compound C (0.3 g). The resulting compound was confirmed by ¹H-NMR.

¹H-NMR (500 MHz, CD₂Cl₂): δ 7.81-7.77(dd, 2H), 7.68(m, 4H), 7.65-7.62(dd, 1H), 7.54(m, 2H), 7.47(m, 4H), 7.37(m, 2H), 7.18(m, 1H), 6.7(d, 1H), 6.65(m, 1H).

### Synthesis Example 4: Synthesis of Compound D

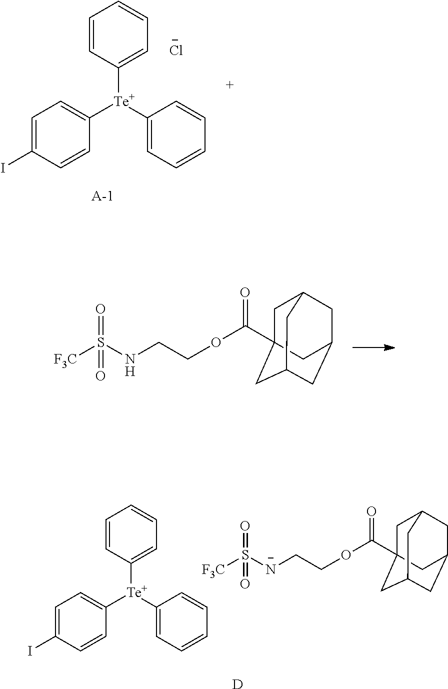

Compound D was obtained in the same manner as used to synthesize Compound C, except that 2-((trifluoromethyl) sulfonamido)ethyl (3r,5r,7r)-adamantane-1-carboxylate was used instead of salicylic acid. 2-((trifluoromethyl) sulfonamido)ethyl (3r,5r,7r)-adamantane-1-carboxylate was synthesized by referring to US Patent Publication US20120214101. The resulting compound was confirmed by ¹H-NMR.

¹H-NMR (500 MHz, CD₂Cl₂): δ 7.85(d, 2H), 7.64-7.60(m, 6H), 7.60-7.53(m, 4H), 7.35(m, 2H), 3.45(t, 2H), 2.75(t, 2H), 1.93(s, 3H), 1.74(s, 6H), 1.71(m, 6H).

### Synthesis Example 5: Synthesis of Polymer X

Polymer X was synthesized with reference to KR20220074627A. Specifically, 0.94 g of dimethyl 2,2′-azobis(2-methylpropionate) (Waco Chemicals), 3.03 g of 2-ethyl-2-adamantyl methacrylate (TCI Chemicals), and 1.98 g of 4-acetoxy styrene (Sigma-Aldrich) were dissolved in tetrahydrofuran, and then, polymerization was performed thereon at a temperature of 80° C. for 8 hours to obtain Polymer X. Polymer X has the following structure, where x and y are each 50.

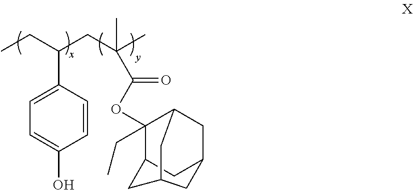

### Evaluation Example 1: Calculation of Transmittance and Absorbance

Assuming a polymer density of 1.20 g/cm³ and a film thickness of 50 nm, the transmittance (T) was calculated by using Website of the Center for X-ray Optics at Lawrence Berkeley National Laboratory. In addition, absorbance (A) was calculated using Equation 1 below. The results are shown in Table 1 below.

A=2−log(% T) [Equation 1]

TABLE 1

| | Cation part | Transmittance (T, %) | Absorbance (A) |
|---|---|---|---|
| Example 1 | 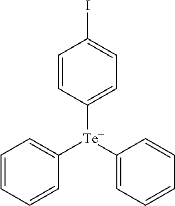 | 65 | 0.19 |
| Comparative Example 1 | 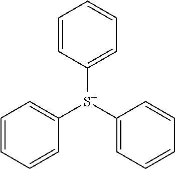 [SMILES: c1ccc([S+](c2ccccc2)c2ccccc2)cc1] | 85.14 | 0.07 |
| Comparative Example 2 | 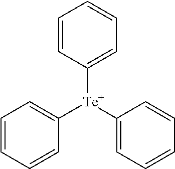 | 70.5 | 0.15 |
| Comparative Example 3 | 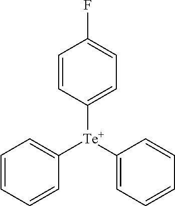 | 69.5 | 0.16 |

From Table 1, it was confirmed that PAG A of Example 1 has lower transmittance and higher absorbance for EUV than those of Comparative Examples 1 to 3.

### Evaluation Example 2: Dose-to-Clear (DCT) Evaluation

Polymer X was dissolved in a 7/3 (wt/wt) solution of propylene glycol methyl ether/propylene glycol methyl ether acetate (PGME/PGMEA), and Compound A was added thereto as a photoacid generator. The obtained solution was spin-coated on a silicon wafer at 1,500 rpm for 60 seconds. EUV exposure was performed after prebaking at 130° C. for 60 seconds. Post-exposure bake was performed at 90° C. for 60 seconds, and then washing was performed using 2.38 wt % TMAH aqueous solution, and the thickness of the remaining coating layer was measured using a three-dimensional optical profiler (Bruker, Contour X-100), from which E₅₀ was calculated through Equation 2 below and shown in Table 2 below.

$$NRT = \frac{d}{{d}_{0}} = {[ 1 + {10}^{log ( E / {E}_{50} ) \times γ} ]}^{- 1} \qquad Equation 2$$

In Equation 2, NRT is the thickness, which is the normalized remaining thickness, and d and do respectively represent the thickness of the coating layer and the maximum thickness of the coating layer according to the irradiation dose (do corresponds to NRT 1). In addition, E is the irradiation dose, and E₅₀ is the irradiation dose when the thickness of the coating layer is half of the maximum thickness (corresponding to NRT 0.5), referring to the sensitivity of the photoresist, and y is the contrast of the photoresist.

TABLE 2

| | E₅₀ |
|---|---|
| | (mJ/cm²) |
| Example 1 | 27.9 |
| Comparative Example 1 | 32.6 |

Referring to Table 2, it can be seen that the photoresist composition of Example 1 has improved sensitivity during EUV exposure, compared to the photoresist composition of Comparative Example 1.

Example embodiments of the present disclosure may provide: an organic salt which may act as a photoacid generator capable of providing improved sensitivity and/or resolution and/or a quencher having improved dispersibility and/or compatibility; and a photoresist composition including the same.

It should be understood that the example embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope as defined by the following claims.

What is claimed is:

1. An organic salt represented by Formula 1: 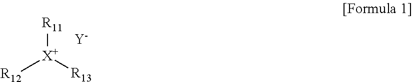
wherein, in Formula 1,
X⁺ is Se⁺ or Te⁺;
R₁₁ to R₁₃ are each independently a linear, branched, or cyclic C₁-C₂₀ monovalent hydrocarbon group which optionally includes a heteroatom;
at least one of R₁₁ to R₁₃ includes at least one iodine (I);
adjacent two of R₁₁ to R₁₃ are optionally bonded to each other to form a ring; and
Y⁻ is a counter anion.

2. The organic salt of claim 1, wherein
X⁺ is Te⁺.

3. The organic salt of claim 1, wherein
R₁₁ to R₁₃ are each independently a C₁-C₂₀ alkyl group, a C₃-C₂₀ cycloalkyl group, or a C₆-C₂₀ aryl group, and
R₁₁ to R₁₃ are each independently unsubstituted or substituted with deuterium, halogen, a cyano group, a hydroxyl group, a carboxyl group, an ester moiety, an ester sulfate moiety, a carbonate moiety, a lactone moiety, a sultone moiety, a carboxylic anhydride moiety, a C₁-C₂₀ alkyl group, a C₁-C₂₀ halogenated alkyl group, a C₁-C₂₀ alkoxy group, a C₃-C₂₀ cycloalkyl group, a C₆-C₂₀ aryl group, or a combination thereof.

4. The organic salt of claim 1, wherein
at least one of R₁₁ to R₁₃ is a C₆-C₂₀ aryl group substituted with at least one I.

5. The organic salt of claim 1, wherein
only one of R₁₁, R₁₂, or R₁₃ includes at least one I;
R₁₁ and R₁₂ each include at least one I, and R₁₃ does not include I; or
R₁₁ to R₁₃ each include at least one I.

6. The organic salt of claim 1, wherein
the organic salt includes one I, two I, or three I.

7. The organic salt of claim 1, wherein
the organic salt is represented by Formula 1-1: 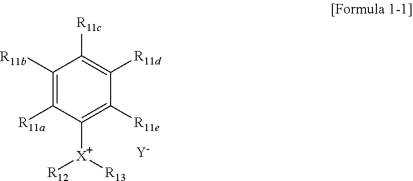
wherein, in Formula 1-1,
R₁₁ₐ to R₁₁ₑ are each independently hydrogen, halogen, or a linear, branched, or cyclic C₁-C₂₀ monovalent hydrocarbon group which optionally includes a heteroatom,
at least one of R₁₁ₐ to R₁₁ₑ, R₁₂, and R₁₃ includes at least one I, and
adjacent two of R₁₁ₐ to R₁₁ₑ, R₁₂, and R₁₃ are optionally bonded to each other to form a ring.

8. The organic salt of claim 7, wherein
at least one of R₁₁ₐ to R₁₁ₑ is I.

9. The organic salt of claim 1, wherein
the organic salt is represented by Formula 1-11 or Formula 1-12: 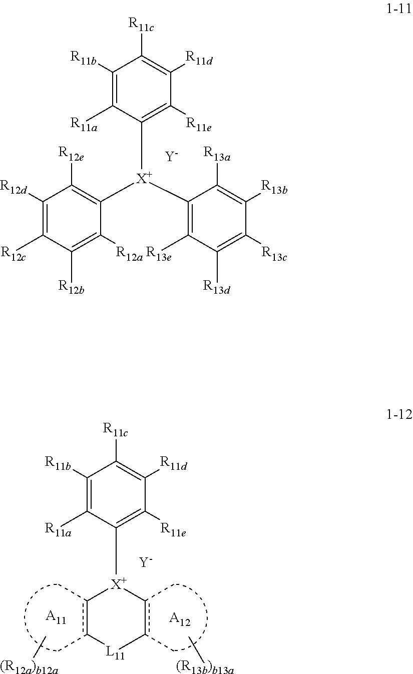
wherein, in Formulae 1-11 and 1-12,
R₁₁ₐ to R₁₁ₑ, R₁₂ₐ to R₁₂ₑ, and R₁₃ₐ to R₁₃ₑ are each independently: hydrogen; halogen; or a linear, branched or cyclic C₁-C₂₀ monovalent hydrocarbon group which optionally includes a heteroatom,
at least one of Rii a to Rule is I,
b12a and b13a are each an integer from 1 to 4,
A₁₁ and Au are each absent or a benzene ring,
L₁₁ is a single bond, O, S, CO, SO, SO₂, CRR′, or NR,
R and R′ are each independently hydrogen, deuterium, halogen, a hydroxyl group, a C₁-C₂₀ alkyl group, a C₁-C₂₀ halogenated alkyl group, a C₁-C₂₀ alkoxy group, a C₃-C₂₀ cycloalkyl group, or a C₃-C₂₀ cycloalkoxy group, and
adjacent two of R₁₁ₐ to R₁₁ₑ, R₁₂ₐ to R₁₂ₑ, and R₁₃ₐ to R₁₃ₑ are optionally linked to each other to form a condensed ring.

10. The organic salt of claim 1, wherein
Y⁻ is represented by at least one of Formulae 2-1 to 2-4: 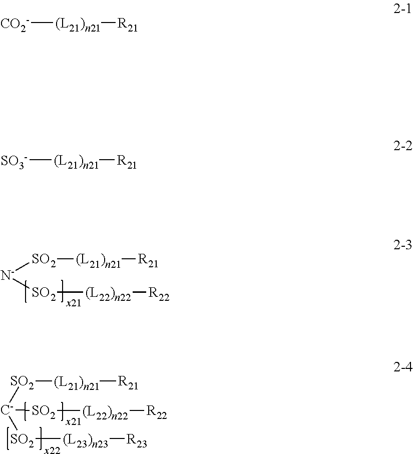
wherein, in Formulae 2-1 to 2-4,
L₂₁ to L₂₃ are each independently a single bond or CRR′,
R and R′ are each independently hydrogen, deuterium, halogen, a hydroxyl group, a C₁-C₂₀ alkyl group, a C₁-C₂₀ halogenated alkyl group, a C₁-C₂₀ alkoxy group, a C₃-C₂₀ cycloalkyl group, or a C₃-C₂₀ cycloalkoxy group,
n21 to n23 are each independently 1, 2, or 3,
x21 and x22 are each independently 0 or 1, and
R₂₁ to R₂₃ are each independently: hydrogen; halogen; or a linear, branched, or cyclic C₁-C₂₀ monovalent hydrocarbon group which optionally includes a heteroatom.

11. The organic salt of claim 1, wherein
Y⁻ is represented by one of Formulae 2-21 to 2-23: 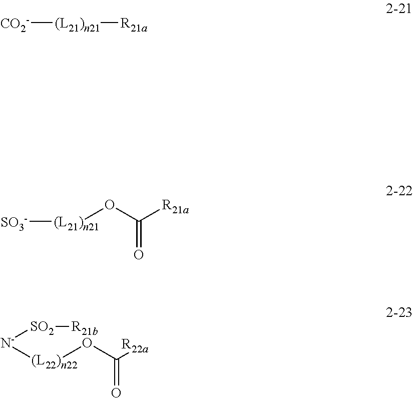
wherein, in Formulae 2-21 to 2-23,
L₂₁ and L₂₂ are each independently a single bond or CXₐX_{b},
Xₐ and X_{b} are each independently hydrogen, halogen, a C₁-C₂₀ alkyl group, or a C₁-C₂₀ halogenated alkyl group,
n21 and n22 are each independently 1, 2, or 3,
R₂₁ₐ, R₂₂ₐ, and R_{21b} are each independently: a C₁-C₂₀ alkyl group, a C₁-C₂₀ halogenated alkyl group, a cyclohexyl group, an adamantyl group, a norbornyl group, a tricyclodecanyl group, a tetracyclododecanyl group, a phenyl group, or a naphthyl group, each unsubstituted or substituted with halogen, a hydroxyl group, a C₁-C₂₀ alkyl group, a C₁-C₂₀ halogenated alkyl group, a C₃-C₂₀ cycloalkyl group, a C₆-C₂₀ aryl group, a C₃-C₂₀ heteroaryl group, or a combination thereof.

12. The organic salt of claim 1, wherein
the organic salt represented by Formula 1 is selected from Group I:

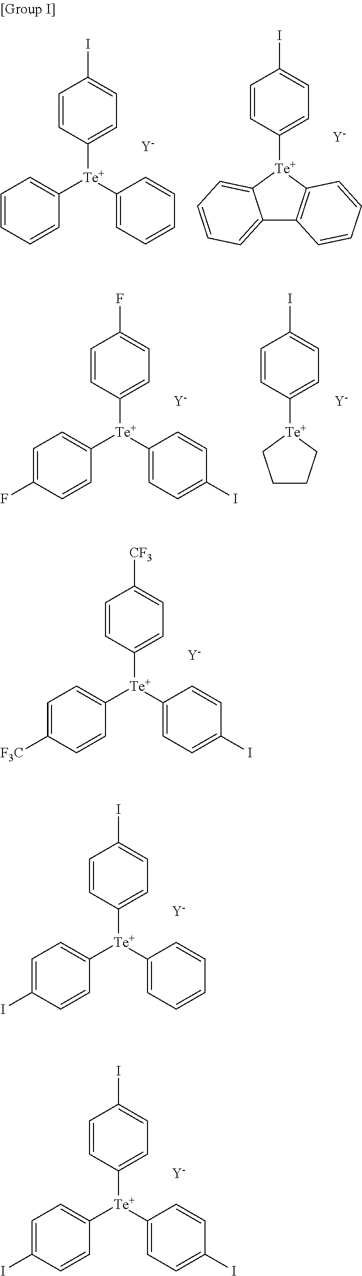

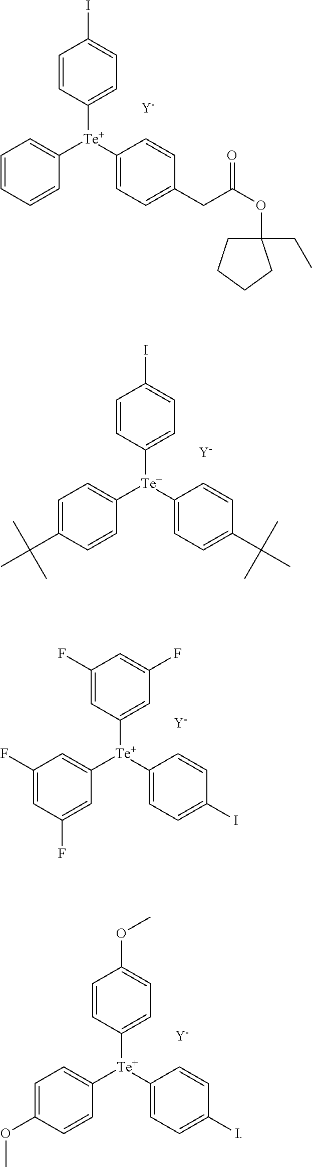

13. The organic salt of claim 12, wherein
Y⁻ is represented by one of Formulae 2-21 to 2-23: 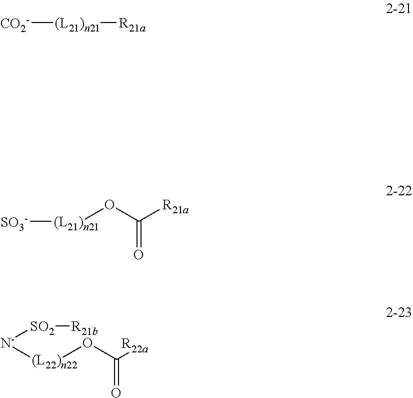
wherein, in Formulae 2-21 to 2-23,
L₂₁ and L₂₂ are each independently a single bond or CXₐX_{b};
Xₐ and X_{b} are each independently hydrogen, halogen, a C₁-C₂₀ alkyl group, or a C₁-C₂₀ halogenated alkyl group,
n21 and n22 are each independently 1, 2, or 3,
R₂₁ₐ, R₂₂ₐ, and R_{21b} are each independently: a C₁-C₂₀ alkyl group, a C₁-C₂₀ halogenated alkyl group, a cyclohexyl group, an adamantyl group, a norbornyl group, a tricyclodecanyl group, a tetracyclododecanyl group, a phenyl group, or a naphthyl group, each unsubstituted or substituted with halogen, a hydroxyl group, a C₁-C₂₀ alkyl group, a C₁-C₂₀ halogenated alkyl group, a C₃-C₂₀ cycloalkyl group, a C₆-C₂₀ aryl group, a C₃-C₂₀ heteroaryl group, or a combination thereof.

14. A photoresist composition comprising:
the organic salt of claim 1;
an organic solvent; and
a base resin.

15. The photoresist composition of claim 14, wherein
the organic salt is configured to be a photodegradable compound that generates an acid upon when exposed to light.

16. The photoresist composition of claim 14, further comprising:
a quencher.

17. The photoresist composition of claim 14, further comprises:
a photoacid generator configured to generate an acid when exposed to light,
wherein the organic salt is configured to be a photodegradable compound that generates an acid when exposure to light and, before the exposure, to act as a quenching base to neutralize the acids.

18. The photoresist composition of claim 14, wherein
an amount of the organic salt is about 0.1 parts by weight to about 40 parts by weight based on 100 parts by weight of the base resin.

19. A pattern formation method comprising:
forming a photoresist film by applying the photoresist composition of claim 14 on a substrate;
exposing at least a portion of the photoresist film to high energy rays; and
developing an exposed photoresist film using a developing solution.

20. The pattern formation method of claim 19, wherein
the exposing includes irradiating ultraviolet (UV), deep ultraviolet (DUV), extreme ultraviolet (EUV), and/or electron beam (EB) rays.
